(19) United States
(12) Patent Application Publication
(10) Pub. No.: US 2017/0355785 A1
(43) Pub. Date: Dec. 14, 2017

(54) PEROXIDE VULCANIZATION OF RUBBER LATEXES

(71) Applicant: Arkema Inc., King of Prussia, PA (US)

(72) Inventors: Peter R. Dluzneski, Harleysville, PA (US); Leonard H. Palys, Downingtown, PA (US); William P. Pavlek, Stevens, PA (US)

(73) Assignee: Arkema Inc., King of Prussia, PA (US)

(21) Appl. No.: 15/520,900

(22) PCT Filed: Oct. 27, 2015
(86) PCT No.: PCT/US2015/057475

§ 371 Date: Apr. 21, 2017

Related U.S. Application Data
(60) Provisional application No. 62/069,871, filed on Oct. 29, 2014.

(51) Int. Cl. C08C 19/22; A61L 31/04; C08C 19/04; C08K 5/14; C08J 3/24; A63H 27/10; C08K 5/29

(52) U.S. Cl. CPC C08C 19/22; A63H 27/10; A61L 31/049; C08K 5/29; C08K 5/14; C08J 3/24; C08C 19/04; A63H 2027/1025; C08J 2309/10

(57) ABSTRACT

A peroxide formulation includes at least one peroxide and at least one compound having a secondary amine group selected from amino acids, such as arginine, folic acid, and polyethyleneamines. The peroxide formulation is capable of curing an aqueous elastomer such as a latex in the full or partial presence of oxygen. Methods of using the peroxide formulation include dip-molding latex elastomer compositions.

### FIELD OF THE INVENTION

The present invention relates to compositions and methods for crosslinking elastomers in the presence of atmospheric oxygen and to products made by those methods.

### BACKGROUND OF THE INVENTION

Elastomers crosslinked with peroxides are known to have superior properties, particularly compared to elastomers crosslinked by sulfur cure. These properties include greater heat stability, better compression set, and no requirement for zinc salts or accelerators to achieve vulcanization. The accelerators that are required for sulfur crosslinking have been known to yield type IV allergies, and the presence of zinc salts typically leads to opacity in the final cured product. In view of its beneficial properties, peroxide cure has a great deal of practical importance. A possible drawback of peroxide curing dip-molded articles is that such articles are commonly dried and cured in hot air ovens or tunnels. The presence of air during peroxide crosslinking is known to lead to tacky surfaces.

In many cases, manufacturers would like to switch from sulfur to peroxide cure and use existing hot air ovens or tunnels; however, curing with conventional peroxide systems under these circumstances would not be viable, as a tacky surface would result. In order to avoid tacky surfaces on objects fabricated using such free radical crosslinking by peroxides, it has been conventional to exclude air from contact with the surface during cure. Measures to exclude oxygen add to the cost and complexity of the cure step and it is often difficult to ensure the complete exhaustion of air and oxygen.

In order to reduce the cost and complexity of the cure step, various methods have been suggested for preventing surface cure inhibition by oxygen during free radical crosslinking. These methods have, for various reasons, met with little or no success. In particular, none have provided a tack-free surface while providing the desirable physical properties of peroxide cure. Moreover, various methods involving sulfur cure and peroxide cure are limited to unsaturated elastomers.

Thus, it is desirable to have peroxide formulations and methods which cure commercially available crosslinkable elastomers, both saturated and unsaturated, in the full or partial presence of atmospheric oxygen.

### SUMMARY OF THE INVENTION

Embodiments of the present invention relate to peroxide formulations that can cure elastomers in the full or partial presence of oxygen (e.g., using a hot air oven or tunnels). Embodiments of the invention also relate to compositions containing the crosslinkable elastomers, processes for curing the elastomers, and products made by such processes.

The applicants have discovered that peroxide formulations containing at least one compound with a secondary amine functionality, particularly amino acids, folic acid, and organic secondary amines having a secondary amine group (such as polyethyleneamines), can significantly reduce the surface tackiness of an elastomeric article that is peroxide cured in the full or partial presence of oxygen. For example, it was surprisingly found that peroxide formulations containing arginine can virtually eliminate the surface tackiness of an elastomeric article that is peroxide cured in an open air system.

Embodiments of the present invention relate to a peroxide formulation comprising, consisting essentially of, or consisting of at least one peroxide and at least one compound having a secondary amine group (e.g., at least one amino acid, such as arginine). The amounts of the at least one peroxide and the at least one compound having a secondary amine group are selected such that the formulation is capable of curing an elastomer composition in the full or partial presence of oxygen (e.g., using a hot air oven or tunnel). According to particular embodiments, the peroxide formulation is in the form of an emulsion, which may further include one or more surfactants.

Embodiments of the present invention also relate to an elastomer composition comprising, consisting essentially of, or consisting of at least one elastomer; at least one peroxide; and at least one compound having a secondary amine group (e.g., at least one amino acid, such as arginine), wherein the elastomer composition is curable in the full or partial presence of oxygen.

Embodiments of the present invention also relate to a process for curing an elastomeric mixture, said process comprising, consisting essentially of, or consisting of curing an elastomeric mixture in the presence of oxygen, wherein the elastomeric mixture comprises, consists essentially of, or consists of at least one elastomer, at least one peroxide and at least one compound having a secondary amine group (e.g., at least one amino acid, such as arginine). Embodiments of the present invention also relate to products made by the above process.

### DETAILED DESCRIPTION

One aspect of the present invention relates to a peroxide formulation comprising, consisting essentially of, or consisting of at least one peroxide and at least one compound having a secondary amine group (e.g., at least one amino acid, such as arginine). As used herein, a compound having a “secondary amine group” or “secondary amine functionality” has at least one nitrogen atom bound to two organic substituents (alkyl, aryl or both) and one hydrogen. The applicants have discovered that, by including one or more compounds having a secondary amine group (e.g., an amino acid, folic acid, and/or an organic secondary amine, such as an polyethyleneamine) in a peroxide formulation, significant reductions in surface tackiness can be obtained when curing elastomers in the full or partial presence of oxygen (e.g., using a hot air oven or tunnel). Therefore, peroxide compositions containing one or more compounds having a secondary amine group can replace sulfur vulcanization in cure processes where oxygen (e.g., atmospheric oxygen) may be present in various amounts. The compositions and methods of the present invention are preferably directed to, and used in conjunction with, liquid elastomers (such as latexes) instead of solid elastomers (such as solid rubbers).

Elastomers that are cured using peroxide compositions of the present invention may include unsaturated elastomers, saturated elastomers, or combinations thereof, whereas sulfur cure and several types of peroxide cure are generally limited to unsaturated elastomers. Thus, embodiments of the invention are not limited by the unsaturation level of elastomers. Moreover, particular embodiments of the invention do not require and may exclude certain components, such as bis-, tri- or higher poly-maleimides, bis-, tri- or higher poly-citraconimides, or silicone elastomers.

According to an embodiment of the present invention, the peroxide formulation comprises, consists essentially of, or consists of at least one peroxide; and at least one compound having a secondary amine group. According to particular embodiments, the compound(s) having a secondary amine group are selected from amino acids folic acid, and organic secondary amines (e.g., polyethyleneamines). For example, the compound(s) having a secondary amine group may include one or more amino acids. The peroxide(s), the compound(s) having a secondary amine group, and their respective amounts, are preferably selected such that the formulation is capable of curing an elastomer composition in the full or partial presence of oxygen (e.g., using a hot air oven or tunnel). Preferably, the formulation is capable of providing a substantially tack-free elastomer composition.

According to particular embodiments, the peroxide formulation comprises, consists essentially of, or consists of:

about 40 wt % to about 60 wt % peroxide(s) (e.g., Luperox® 26, which is t-butylperoxy 2-ethylhexanoate, sold by Arkema, Inc.),

about 10 wt % to about 30 wt % compound(s) having a secondary amine group (e.g., arginine),

about 20 wt % to about 35 wt % water, and

about 0.1 wt % to about 5 wt % optional surfactant(s).

According to further embodiments, the peroxide formulation comprises, consists essentially of, or consists of:

about 50 wt % peroxide(s) (e.g., Luperox® 26, which is t-butylperoxy 2-ethylhexanoate, sold by Arkema, Inc.),

about 20 wt % compound(s) having a secondary amine group (e.g., arginine),

about 28 wt % water, and

about 2 wt % optional surfactant(s).

According to particular embodiments, the peroxide formulation comprises, consists essentially of, or consist of at least one peroxide selected from the group consisting of t-butylperoxy 2-ethyhexanoate, tert-amyl peroxy-2-ethyhexylcarbonate, and aqueous dibenzoyl peroxide, and at least one compound selected from the group consisting of arginine and folic acid.

According to particular embodiments, the peroxide formulation is capable of curing an elastomer composition at one or more temperatures between about 110° C. and about 130° C. in an amount of time that is between about 8 minutes and about 30 minutes.

All those organic peroxides known to undergo decomposition by heat to generate radicals capable of initiating the desired curing (crosslinking) reactions are contemplated as suitable for use in the present invention. Non-limiting examples include dialkyl peroxides, peroxyketals, monoperoxy carbonates, ketone peroxides, diacyl peroxides, organosulfonyl peroxides, peroxyesters, peroxydicarbonates, hydroperoxides and diacyl peroxides.

Peroxide names and physical properties for all these classes of organic peroxides can be found in “Organic Peroxides” by Jose Sanchez and Terry N. Myers; Kirk-Othmer Encyclopedia of Chemical Technology, Fourth Ed., Volume 18, (1996), the disclosure of which is incorporated herein by reference.

Illustrative dialkyl peroxide initiators include:
di-t-butyl peroxide;
t-butyl cumyl peroxide;
2,5-di(cumylperoxy)-2,5-dimethyl hexane;
2,5-di(cumylperoxy)-2,5-dimethyl hexyne-3;
4-methyl-4-(t-butylperoxy)-2-pentanol;
4-methyl-4-(t-amylperoxy)-2-pentanol;
4-methyl-4-(cumylperoxy)-2-pentanol;
4-methyl-4-(t-butylperoxy)-2-pentanone;
4-methyl-4-(t-amylperoxy)-2-pentanone;
4-methyl-4-(cumylperoxy)-2-pentanone;
2,5-dimethyl-2,5-di(t-butylperoxy)hexane;
2,5-dimethyl-2,5-di(t-amylperoxy)hexane;
2,5-dimethyl-2,5-di(t-butylperoxy)hexyne-3;
2,5-dimethyl-2,5-di(t-amylperoxy)hexyne-3;
2,5-dimethyl-2-t-butylperoxy-5-hydroperoxyhexane;
2,5-dimethyl-2-cumylperoxy-5-hydroperoxy hexane;
2,5-dimethyl-2-t-amylperoxy-5-hydroperoxyhexane;
m/p-alpha, alpha-di[(t-butylperoxy)isopropyl]benzene;
1,3,5-tris(t-butylperoxyisopropyl)benzene;
1,3,5-tris(t-amylperoxyisopropyl)benzene;
1,3,5-tris(cumylperoxyisopropyl)benzene;
di[1,3-dimethyl-3-(t-butylperoxy)butyl]carbonate;
di[1,3-dimethyl-3-(t-amylperoxy)butyl]carbonate;
di[1,3-dimethyl-3-(cumylperoxy)butyl]carbonate;
di-t-amyl peroxide;
t-amyl cumyl peroxide;
2,4,6-tri(butylperoxy)-s-triazine;
1,3,5-tri[1-(t-butylperoxy)-1-methylethyl]benzene
1,3,5-tri-[(t-butylperoxy)-isopropyl]benzene;
1,3-dimethyl-3-(t-butylperoxy)butanol;
1,3-dimethyl-3-(t-amylperoxy)butanol; and mixtures thereof.

Illustrative solid, room temperature stable peroxydicarbonates include, but are not limited to:

di(2-phenoxyethyl)peroxydicarbonate; di(4-t-butyl-cyclohexyl)peroxydicarbonate; dimyristyl peroxydicarbonate; dibenzyl peroxydicarbonate; and di(isobornyl)peroxydicarbonate.

Another class of dialkylperoxides which may be used singly or in combination with the other free radical initiators contemplated by the present disclosure are those selected from the group represented by the formula:

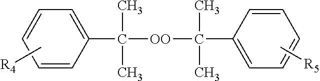

wherein R₄ and R₅ may independently be in the meta or para positions and are the same or different and are selected from hydrogen or straight or branched chain alkyls of 1 to 6 carbon atoms. Dicumyl peroxide and isopropylcumyl cumyl peroxide are illustrative.

Other dialkyl peroxides include:
3-cumylperoxy-1,3-dimethylbutyl methacrylate;
3-t-butylperoxy-1,3-dimethylbutyl methacrylate;
3-t-amylperoxy-1,3-dimethylbutyl methacrylate;
tri(1,3-dimethyl-3-t-butylperoxy butyloxy)vinyl silane;
1,3-dimethyl-3-(t-butylperoxy)butyl N-[1-{3-(1-methylethenyl)-phenyl}1-methylethyl]carbamate;
1,3-dimethyl-3-(t-amylperoxy)butyl N-[1-{3-(1-methylethenyl)-phenyl}-1-methylethyl]carbamate;
1,3-dimethyl-3-(cumylperoxy))butyl N-[1-{3-(1-methylethenyl)-phenyl}-1-methylethyl]carbamate.

In the group of peroxyketal initiators, the preferred initiators include:
1,1-di(t-butylperoxy)-3,3,5-trimethylcyclohexane;
1,1-di(t-butylperoxy)cyclohexane;
n-butyl 4,4-di(t-amylperoxy)valerate;
ethyl 3,3-di(t-butylperoxy)butyrate;
2,2-di(t-amylperoxy)propane;
3,6,6,9,9-pentamethyl-3-ethoxycabonylmethyl-1,2,4,5-tetraoxacyclononane;
n-butyl-4,4-bis(t-butylperoxy)valerate;
ethyl-3,3-di(t-amylperoxy)butyrate; and mixtures thereof.

Other peroxides that may be used according to at least one embodiment of the present disclosure include benzoyl peroxide, OO-t-butyl-O-hydrogen-monoperoxy-succinate and OO-t-amyl-O-hydrogen-monoperoxy-succinate.

Illustrative cyclic ketone peroxides are compounds having the general formulae (I), (II) and/or (III).

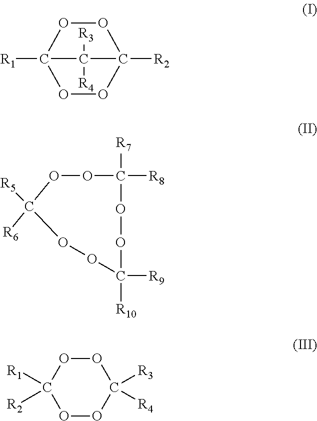

wherein R₁ to R₁₀ are independently selected from the group consisting of hydrogen, C1 to C20 alkyl, C3 to C20 cycloalkyl, C6 to C20 aryl, C7 to C20 aralkyl and C7 to C20 alkaryl, which groups may include linear or branched alkyl properties and each of R₁ to R₁₀ may be substituted with one or more groups selected from hydroxy, C1 to C20 alkoxy, linear or branched C1 to C20 alkyl, C6 to C20 aryloxy, halogen, ester, carboxy, nitride and amido, such as, for example, at least 20% of the total active oxygen content of the peroxide mixture used for a crosslinking reaction will be from compounds having formulas (I), (II) and/or (III).

Some examples of suitable cyclic ketone peroxides include:

3,6,9, triethyl-3,6,9-trimethyl-1,4,7-triperoxynonane (or methyl ethyl ketone peroxide cyclic trimer), methyl ethyl ketone peroxide cyclic dimer, and 3,3,6,6,9,9-hexamethyl-1,2,4,5-tetraoxacyclononane.

Illustrative examples of peroxy esters include:
2,5-dimethyl-2,5-di(benzoylperoxy)hexane;
t-butylperbenzoate;
t-butylperoxy acetate;
t-butylperoxy-2-ethyl hexanoate;
t-amyl perbenzoate;
t-amyl peroxy acetate;
t-butyl peroxy isobutyrate;
3-hydroxy-1,1-dimethyl t-butyl peroxy-2-ethyl hexanoate;
OO-t-amyl-O-hydrogen-monoperoxy succinate;
OO-t-butyl-O-hydrogen-monoperoxy succinate;
di-t-butyl diperoxyphthalate;
t-butylperoxy (3,3,5-trimethylhexanoate);
1,4-bis(t-butylperoxycarbo)cyclohexane;
t-butylperoxy-3,5,5-trimethylhexanoate;
t-butyl-peroxy-(cis-3-carboxy)propionate;
allyl 3-methyl-3-t-butylperoxy butyrate.

Illustrative monoperoxy carbonates include:
OO-t-butyl-O-isopropylmonoperoxy carbonate;
OO-t-butyl-O-(2-ethyl hexyl)monoperoxy carbonate;
1,1,1-tris[2-(t-butylperoxy-carbonyloxy)ethoxymethyl]propane;
1,1,1-tris[2-(t-amylperoxy-carbonyloxy)ethoxymethyl]propane;
1,1,1-tris[2-(cumylperoxy-cabonyloxy)ethoxymethyl]propane;
OO-t-amyl-O-isopropylmonoperoxy carbonate.

Illustrative diacyl peroxides include:
di(4-methylbenzoyl)peroxide;
di(3-methylbenzoyl)peroxide;
di(2-methylbenzoyl)peroxide;
didecanoyl peroxide; dilauroyl peroxide; 2,4-dibromo-benzoyl peroxide;
succinic acid peroxide.
dibenzoyl peroxide (including but not limited to dibenzoyl peroxide in water);
di(2,4-dichloro-benzoyl)peroxide.

Imido peroxides of the type described in PCT Application publication WO9703961 A1 6 Feb. 1997 are also contemplated as suitable for use and incorporated by reference herein.

In at least one embodiment, the peroxide(s) are selected from peroxyesters and peroxyketals. According to particular embodiments, the peroxide(s) are selected from the group consisting of t-butyl peroxy-2-ethylhexanoate (e.g., Luperox® 26, sold by Arkema, Inc.), OO-t-amyl-O-(2-ethylhexyl) monoperoxycarbonate (e.g., Luperox® TAEC, sold by Arkema, Inc.), 1,1-di-(t-amylperoxy)cyclohexane (e.g., Luperox® 531M80, sold by Arkema, Inc.), and a combination thereof.

Embodiments of the peroxide formulations of the present invention may include at least one amino acid having at least one secondary amine group. In addition to one or more secondary amine groups, the amino acid may contain one or more other types of nitrogen-containing functional groups, such as primary amine groups and/or imine groups. The secondary amine group(s) may be part of a heterocyclic ring, e.g., an imidazole ring. Non-limiting examples of amino acids that may be included in peroxide formulations of the present invention include arginine, proline, hydroxyproline, and histidine. According to particular embodiments, the amino acid(s) are naturally occurring. In exemplary embodiments, the amino acid(s) comprise, consist essentially of, or consist of arginine.

According to alternative embodiments, the peroxide formulation of the present invention includes one or more organic secondary amines, such as polyethyleneamines having one or more secondary amine groups; for example, tetraethylenepentamine (TEPA), triethylenetetramine (TETA) and/or diethylenetriamine (DETA). According to these embodiments, the peroxide formulation may comprise, consist essentially of, or consist of at least one peroxide and one or more polyethyleneamines selected from the group consisting of tetraethylenepentamine (TEPA), triethylenetetramine (TETA) and diethylenetriamine (DETA). The polyethyleneamine may correspond to the general structure H₂N(CH₂CH₂NH)ₙH wherein n=2-6, for example.

According to alternative embodiments, the peroxide formulation of the present invention may include one or more compounds having at least one secondary amine group, wherein the one or more compounds are selected from the group consisting of: amino acids having at least one secondary amine group, folic acid, polyethyleneamines having at least one secondary amine group, and a combination thereof. For example, the one or more compounds may be selected from the group consisting of arginine, proline, hydroxyproline, histidine, folic acid, TEPA, TETA, DETA, and a combination thereof.

Organic peroxide formulations of the present invention may be prepared in the form of a liquid. For example, an amino acid (e.g., arginine), folic acid, or polyethyleneamine having a secondary amine functionality may be dissolved in a water-based solution (preferably water) and combined with a liquid peroxide. According to at least one embodiment, a liquid peroxide formulation of the present invention is in the form of an emulsion. For example, the emulsion may comprise at least one peroxide (e.g., a peroxyester and/or peroxyketal, such as t-butyl peroxy-2-ethylhexanoate, OO-t-amyl-O-(2-ethylhexyl) monoperoxycarbonate, and/or 1,1-di-(t-amylperoxy)cyclohexane) emulsified in an aqueous solution that contains an amino acid or an polyethyleneamine having a secondary amine functionality (e.g., arginine, proline, hydroxyproline, histidine, folic acid, TEPA, TETA or DETA). This emulsion may then be blended with an elastomer, or a mixture of elastomers, prior to curing. Alternatively, the peroxide(s) may first be added to the elastomer(s), followed by the amino acid, folic acid, or polyethyleneamine, prior to curing.

The organic peroxide formulation may further include one or more surfactants, particularly when the formulation is in the form of an emulsion. Non-limiting examples of surfactants include sorbitan esters, partially hydrolyzed polyvinyl acetate, ethoxylated fatty acid salts, ethoxylated fatty alcohols, n-alkylbenzenesulfonic acid salts and fatty acid salts.

Organic peroxide formulations of the present invention may alternatively be prepared in the form of a solid. For example, a liquid peroxide formulation that includes at least one peroxide emulsified in an aqueous solution of an amino acid, folic acid, or polyethyleneamine may be adsorbed onto an inert filler, such as by spraying.

According to particular embodiments, the peroxide formulation of the present invention comprises, consists essentially of, or consists of at least one organic peroxide; at least one amino acid, folic acid, or polyethyleneamine having a secondary amine group (e.g., arginine); at least one optional surfactant; and at least one optional filler; wherein the amounts of each of the components are selected such that the formulation is capable of curing an elastomer composition in the full or partial presence of oxygen. Preferably, the formulation is capable of providing a substantially tack-free elastomer composition.

Another aspect of the present invention relates to an elastomer composition (also referred to herein as an elastomeric mixture) comprising, consisting essentially of, or consisting of at least one elastomer; at least one peroxide; at least one compound having a secondary amine functionality, such as an amino acid, folic acid, or an organic secondary amine (e.g., an polyethyleneamine); and at least one optional surfactant, wherein the elastomer composition is curable in the full or partial presence of oxygen

In at least one embodiment, the elastomer composition may comprise a saturated elastomer, an unsaturated elastomer, or both a saturated and unsaturated elastomer; for example, elastomer compositions may include, but are not limited to, latexes, water-based latexes, or solvent-based latexes, such as natural rubber latex, synthetic rubber latex, and the like. According to preferred embodiments, the elastomer is not solid rubber, but is liquid (e.g., liquid latex).

It should be noted that commercially-available pre-compounded elastomers may be used in accordance with the present invention. These elastomers may contain additives such as carbon black filler, process oils, mold release agents, antioxidants and/or heat stabilizers.

According to at least one embodiment, the elastomer composition comprises at least one saturated elastomer. The saturated elastomer can be selected from, for example, fluoroelastomers (e.g., FKM), chlorinated polyethylene, hydrogenated nitrile butadiene (HNBR), ethylene-vinyl acetate (EVA), ethylene-propylene rubber (EPM), ethylene-butene rubber (EBM), ethylene-octene rubber (EOM), and combinations thereof.

According to at least one embodiment, the elastomer composition comprises at least one unsaturated elastomer. Unsaturated elastomers that may be used in the elastomer composition include, for example, natural rubber (NR), nitrile rubber (NBR), carboxylated nitrile rubber (XNBR), styrene butadiene rubber (SBR), synthetic polyisoprene rubber (IR), neoprene rubber (CR), butadiene rubber (BR), ethylene-propylene-diene rubber (EPDM), styrene-ethylene-butylene-styrene rubber (SEBS) and combinations thereof.

At least one embodiment of the present invention relates to a method for manufacturing an article comprising an elastomer composition as described herein, wherein the method comprises curing the elastomer composition in the full or partial presence of oxygen (e.g., using a hot air oven or tunnel).

As used herein, the term “curing” refers to the crosslinking of polymer chains to form a strengthened or hardened polymer. A curing, or crosslinking, step may be performed in any conventional manner, such as, for example, hot air or hot molding. The method for manufacturing the article may be performed in a hot air oven or tunnel, or any other known apparatus.

An additional embodiment of the present invention relates to a process for curing an elastomeric mixture, the process comprising, consisting essentially of, or consisting of curing the elastomeric mixture in the full or partial presence of oxygen, wherein the elastomeric mixture comprises, consists essentially of, or consists of at least one elastomer, at least one peroxide, and at least one compound having a secondary amine functionality, such as an amino acid, folic acid, or a polyethyleneamine. The process may further comprise mixing or blending the at least one elastomer, the at least one peroxide, and at least one compound having a secondary amine functionality to provide the elastomeric mixture, preferably allowing time for the components to disperse evenly.

According to particular embodiments, the process comprises curing the elastomeric mixture in the presence of oxygen at one or more temperatures between about 70° C. and about 150° C. (i.e., the temperature may change one or more times during the curing process).

According to an additional embodiment, the process includes one or more of the following steps after the components of the elastomeric mixture (e.g., peroxide(s), elastomer(s) and compound(s) having secondary amine functionality) have dispersed evenly:

drying the elastomeric mixture in the presence of oxygen (e.g., on a form) at ambient or elevated temperatures to yield a rubber film (e.g., at 20-100° C. for 1-60 min); and heating the dried rubber film in the presence of oxygen (e.g., on a form) to effect the final cure (e.g., at a temperature between 70° C. and 150° C., preferably between 80° C. and 140° C., more preferably between 110° C. and 130° C., for 3 to 120 minutes, preferably for 5 to 60 minutes, more preferably for 7 to 30 minutes). When an article is made by dip-molding, the drying and heating steps are performed while a layer of the elastomeric mixture is on a mold or form that corresponds to the shape of the final article.

In at least one embodiment, conventional additives such as anti-oxidants (e.g., hindered phenols and polymeric quinoline derivatives), aliphatic process oils, and other process aids, pigments, dyes, waxes, reinforcing aids, UV stabilization agents, blowing agents and activators and antiozonants may also be added to the elastomer compositions before curing.

Processes of the present invention may further include dip-molding the above-described elastomer composition. In accordance with these processes, a layer of the elastomer composition is formed on a mold or form (for example, by dipping the mold or form into the elastomer composition), the shape of which corresponds to the shape of the final cured article. Non-limiting examples of dip-molded articles made by such methods include gloves, condoms, balloons, and medical devices such as vial stoppers, bladders, anesthesia bags and bulbs.

As a method of dip-forming, there may be used methods known in the art such as direct dipping method, anode coagulant dipping method, teague coagulant dipping method and the like. For example, a dip-forming mold may be dipped in a coagulant solution (e.g., calcium chloride or calcium nitrate in water, alcohol or a mixture thereof) so that the coagulant adheres to its surface, and then the mold may be dipped in an elastomer composition of the present invention to form a dip-formed rubber layer thereon. As a dip-forming mold, there may be used various molds such as those made of ceramics, glass, metal, plastics or the like. The shape of the mold corresponds to the shape of the final dip-formed article (e.g., a glove, condom, balloon, vial stopper, bladder or bulb). The surface of the dip-forming mold may be wholly or partially surface-treated, such as by glossing, semi-glossing, non-glossing, fabric patterning and the like. The dip-formed rubber layer may be dipped in water (e.g., at a temperature of 30-70° C., for 1-60 min) to remove water-soluble impurities before or after heat treatment.

According to particular embodiments, an elastomer composition of the present invention comprises, consists essentially of, or consists of at least one elastomer (either saturated, unsaturated, or both); at least one peroxide; and at least one compound having a secondary amine functionality (e.g., an amino acid, such as arginine, or a polyethyleneamine), which has been cured in the full or partial presence of oxygen, has less surface tackiness in comparison to an elastomer composition that has been cured according to an identical process and that has an identical composition except that it does not include the at least one compound having secondary amine functionality.

Surface tackiness may be judged, for example, by a “glove touch test” or “facial tissue paper test,” as described in the Examples below.

The embodiments described herein are intended to be exemplary of the invention and not limitations thereof. One skilled in the art will appreciate that modifications to the embodiments and examples of the present disclosure may be made without departing the scope of the present disclosure. The embodiments of the invention are described above using the term “comprising” and variations thereof. However, it is the intent of the inventors that the term “comprising” may be substituted in any of the embodiments described herein with “consisting of” and “consisting essentially of” without departing the scope of the invention.

The following examples further illustrate the best mode contemplated by the inventors for the practice of their invention and are to be construed as illustrative and not in limitation thereof.

### EXAMPLES

### Example 1

A peroxide-cured latex formulation was prepared using the following components:
1. 5 grams Cariflex® IR401 (a latex containing synthetic polyisoprene from Kraton Performance Polymers, Inc.).
2. 50 milligrams Luperox® 26 (t-butylperoxy 2-ethylhexanoate from Arkema, Inc.).
3. 50 milligrams aqueous arginine (33%, pH 10).

The aqueous arginine solution was made by diluting one part of arginine hydrochloride in two parts of deionized water and then adjusting to pH 10 with 50% caustic. The neat peroxide was added directly to the latex dispersion and was allowed to stir for one hour on a magnetic stirrer before the addition of the aqueous arginine solution. After adding the aqueous arginine, the latex was stirred for five minutes before pouring the latex into an aluminum pan. No coagulation of the latex was observed. The latex was then allowed to dry in the open air overnight. After drying, the latex was placed in an open-air oven at 110° C. for thirty minutes. After allowing one minute to cool, the surface was touched using a gloved hand. Samples cured without the arginine had a surface that was visibly tacky. Samples cured with arginine in the formulation gave virtually no tackiness.

### Facial Tissue Paper Test

The following procedure was used to test the surface tack of the rubber sheet after curing in a hot air oven or tunnel. This procedure is also referred to as a “Facial Tissue Paper Test” for surface tackiness of a rubber sheet cured in a hot air oven or tunnel.

After addition of peroxide and other ingredients to the latex, a sample of the latex is poured into a pan and allowed to dry overnight at ambient temperature. This dried latex film is then placed in an oven at 110° C. for thirty minutes to cure. The cured film is then removed from the oven and allowed to cool to ambient temperature for two minutes. After cooling the entire rubber surface is covered by a Kleenex® facial tissue and firm pressure is applied by hand. The facial tissue is then removed and the surface is inspected for tissue residue that may have adhered to the surface. If many tissue paper fibers adhere, this indicates a poor surface cure, or one that has a high amount of surface tackiness.

As used herein, the Surface Tackiness Number=(% of surface with no paper fibers÷10). The Surface Tackiness number can range from 10 to 0. A completely tack-free cured rubber surface with no tissue paper fibers has a rating of 10. A poorly cured rubber surface that is completely covered in tissue paper fibers is rated a 0. If 90% of the surface has no tissue paper fibers attached, the rating is a 9, etc.

### Example 2 (Pan Test)

A peroxide-cured latex formulation was prepared using the following components:
1. 5 grams Cariflex® IR401 (a latex containing synthetic polyisoprene from Kraton Performance Polymers, Inc.).
2. 50 milligrams Luperox® 26 (t-butylperoxy 2-ethylhexanoate from Arkema, Inc.).
3. 50 milligrams aqueous arginine (30%, pH 10).

The aqueous arginine solution was made by diluting arginine hydrochloride in deionized water and then adjusting to pH 10 with 50% caustic to yield a 30% concentration of the arginine hydrochloride. The neat peroxide was added directly to the latex dispersion and was allowed to stir for one hour on a magnetic stirrer before the addition of the aqueous arginine solution. After adding the aqueous arginine, the latex was stirred for five minutes before pouring the latex into an aluminum pan. No coagulation of the latex was observed. The latex was then allowed to dry in the open air overnight. After drying, the latex was placed in an open-air oven at 110° C. for thirty minutes. After allowing one minute to cool, the surface was touched using a gloved hand. Samples cured without the arginine had a surface that was visibly tacky. Samples cured with arginine in the formulation gave virtually no tackiness.

### Example 3 (Pan Test)

A peroxide-cured latex formulation was prepared using the following components:
1. 5 grams Cariflex® IR401 (a latex containing synthetic polyisoprene from Kraton Performance Polymers, Inc.).
2. 50 milligrams Luperox® 26 (t-butylperoxy 2-ethylhexanoate from Arkema, Inc.).
3. 50 milligrams aqueous tetraethylene pentamine (33%).

The aqueous tetraethylene penatmine solution was made by diluting one part of tetraethylene pentaminehydrochloride in two parts of deionized water. The neat peroxide was added directly to the latex dispersion and was allowed to stir for one hour on a magnetic stirrer before the addition of the aqueous tetraethylene pentamine solution. After adding the aqueous tetraethylene pentamine, the latex was stirred for five minutes before pouring the latex into an aluminum pan. No coagulation of the latex was observed. The latex was then allowed to dry in the open air overnight. After drying, the latex was placed in an open-air oven at 110° C. for thirty minutes. After allowing one minute to cool, the surface was touched using a gloved hand. Samples cured without the tetraethylene pentaamine had a surface that was visibly tacky. Samples cured with tetraethylene pentamine in the formulation gave virtually no tackiness.

### Example 4 (Pan Test)

A peroxide-cured latex formulation was prepared using the following components:
1. 5 grams Cariflex® IR401 (a latex containing synthetic polyisoprene from Kraton Performance Polymers, Inc.).
2. 50 milligrams Luperox® TAEC (tert-Amyl peroxy-2-ethylhexylcarbonate from Arkema, Inc.).
3. 50 milligrams aqueous arginine (30%, pH 10).

The aqueous arginine solution was made by diluting arginine hydrochloride in deionized water and then adjusting to pH 10 with 50% caustic to yield a 30% concentration of the arginine hydrochloride. The neat peroxide was added directly to the latex dispersion and was allowed to stir for one hour on a magnetic stirrer before the addition of the aqueous arginine solution. After adding the aqueous arginine, the latex was stirred for five minutes before pouring the latex into an aluminum pan. No coagulation of the latex was observed. The latex was then allowed to dry in the open air overnight. After drying, the latex was placed in an open-air oven at 130° C. for thirty minutes. After allowing one minute to cool, the surface was touched using a gloved hand. Samples cured without the arginine had a surface that was visibly tacky. Samples cured with arginine in the formulation gave virtually no tackiness.

### Example 5 (Dip Mold)

A peroxide-cured latex formulation was prepared using the following components:
1. 403 grams Centex® HA (a natural rubber latex from Centrotrade Inc.).
2. 197 grams of distilled deionized water
3. 36 grams Luperox® A40FP EZ-9 (Dibenzoyl peroxide in water from Arkema, Inc.).
4. 36 grams aqueous arginine (30%, pH 10).

The aqueous arginine solution was made by diluting arginine hydrochloride in deionized water and then adjusting to pH 10 with 50% caustic. The natural rubber latex was added to an enclosed, jacketed kettle equipped with overhead stirring. Heated water was circulated through the kettle jacket to allow for temperature control. Deionized water was added to the latex in the kettle to dilute the solids content to 42% and allowed to mix for one hour. Luperox® A40FP EZ-9 was added slowly to the diluted latex over a period of ten minutes and allowed to stir for thirty minutes. The aqueous arginine was then added slowly over a period of ten minutes. This mixture was stirred at ambient temperature over the course of 7 days with dip samples taken at 24, 48, 72, and 168 hours.

To perform the dip operation, a 16 oz wide mouth glass bottle was used as a form. This bottle was cleaned and coated with an aqueous coagulant solution consisting of 33% calcium nitrate, 66.6% deionized water, and 0.1% Surfonyl® 465 which was obtained from Air Products Inc. The cleaned bottle form was dipped for one minute in this solution and allowed to dry in an oven at 55° C. for ten minutes while being turned horizontally to eliminate pooling. The coagulant-coated bottle form was then dipped in the latex bath for five minutes and then dried in an oven at 55° C. for one hour while being turned horizontally to eliminate pooling. The dried latex-coated form was then placed in another oven set at 110° C. for thirty minutes to effect the cure.

The cured latex samples obtained from this method showed no evidence of surface tackiness. Tensile bars were then cut from these to determine the extent of cure. These data are presented in Table 1.

TABLE 1

| | Results of Tensile Tests | | |
|---|---|---|---|
| | from Dip-Mold Process | | |
| | | Tensile Strength | Elongation |
| Cure Additives | Dip Time | (MPa) | (%) |
| Luperox ® A40FP EZ-9 | 24 hours | 1.61 | 752 |
| (6 phr) with 30% Arginine | 48 hours | 3.40 | 1007 |
| (6 phr) (Ambient Temp) | 72 hours | 3.12 | 1119 |
| | 168 hours | 2.21 | 936 |

### Example 6 (Dip Mold)

A peroxide-cured latex formulation was prepared using the following components:
1. 403 grams Centex® HA (a natural rubber latex from Centrotrade Inc.).
2. 197 grams of distilled deionized water
3. 36 grams Luperox® A40FP EZ-9 (Dibenzoyl peroxide in water from Arkema, Inc.).
4. 36 grams aqueous folic acid (20%, pH 10).

The aqueous folic acid solution was made by diluting folic acid in deionized water and then adjusting to pH 10 with 50% caustic. The natural rubber latex was added to an enclosed, jacketed kettle equipped with overhead stirring. Heated water was circulated through the kettle jacket to allow for control of the temperature at 40° C. Deionized water was added to the latex in the kettle to dilute the solids content to 42% and allowed to mix for one hour. Luperox® A40FP EZ-9 was added slowly to the diluted latex over a period of ten minutes and allowed to stir for thirty minutes. The aqueous folic acid was then added slowly over a period of ten minutes. This mixture was stirred at ambient temperature over the course of 7 days with dip samples taken at 24, 72, and 168 hours.

The cured latex samples obtained from this method showed no evidence of surface tackiness. Tensile bars were then cut from these to determine the extent of cure. These data are presented in Table 2.

TABLE 2

| | Results of Tensile Tests | | |
|---|---|---|---|
| | from Dip-Mold Process | | |
| | | Tensile Strength | Elongation |
| Cure Additives | Dip Time | (MPa) | (%) |
| EZ9 (6 phr) with 20% Folic | 24 hours | 3.28 | 788 |
| Acid (6 phr) (40° C.) | 72 hours | 3.77 | 866 |
| | 168 hours | 2.31 | 741 |

1. A peroxide formulation for curing a latex elastomer composition in the presence of oxygen comprising:
at least one peroxide; and
at least one compound having secondary amine functionality selected from amino acids folic acid, and organic secondary amines.

2. The peroxide formulation of claim 1, wherein the at least one compound having secondary amine functionality is selected from amino acids and polyethyleneamines.

3. The peroxide formulation of claim 1, wherein the amounts of the at least one peroxide and the at least one compound having secondary amine functionality are selected such that the formulation is capable of curing an elastomer composition in the presence of oxygen.

4. The peroxide formulation of claim 1, wherein the at least one compound having secondary amine functionality is selected from the group consisting of arginine, proline, hydroxyproline, histidine, tetraethylenepentamine (TEPA), triethylenetetramine (TETA), diethylenetriamine (DETA), folic acid and a combination thereof.

5. The peroxide formulation of claim 1, wherein the at least one compound having secondary amine functionality comprises one or more amino acids.

6. The peroxide formulation of claim 1, wherein the at least one compound having secondary amine functionality is selected from the group consisting of arginine, proline, hydroxyproline, folic acid and histidine.

7. The peroxide formulation of claim 1, wherein the at least one compound having secondary amine functionality comprises arginine or folic acid.

8. The peroxide formulation of claim 1, wherein the at least one peroxide is selected from peroxyesters and peroxyketals.

9. The peroxide formulation of claim 1, wherein the at least one peroxide is selected from the group consisting of t-butyl peroxy-2-ethylhexanoate, OO-t-amyl-O-(2-ethylhexyl) monoperoxycarbonate, 1,1-di-(t-amylperoxy)cyclohexane, dibenzoyl peroxide, and a combination thereof.

10. The peroxide formulation of claim 1, wherein the at least one peroxide comprises t-butylperoxy 2-ethylhexanoate or dibenzoyl peroxide.

11. The peroxide formulation of claim 1, wherein the peroxide formulation is in the form of an aqueous emulsion.

12. The peroxide formulation of claim 1 further comprising at least one surfactant.

13. The peroxide formulation of claim 1 further comprising an inert filler, wherein the peroxide formulation is in the form of a solid powder.

14. The peroxide formulation of claim 1, wherein the peroxide formulation is in the form of an emulsion.

15. A method for manufacturing the peroxide formulation of claim 1 comprising mixing the at least one peroxide and the at least one compound having secondary amine functionality.

16. A latex elastomer composition comprising:
at least one latex elastomer; and
at least one peroxide; and
at least one compound having secondary amine functionality selected from amino acids and organic secondary amines,
wherein the elastomer composition is curable in the presence of oxygen.

17. The elastomer composition of claim 16, wherein the at least one compound having secondary amine functionality is selected from amino acids, folic acid, and polyethyleneamines.

18. The elastomer composition of claim 16, wherein the amounts of the at least one peroxide and the at least one compound having secondary amine functionality are selected such that the elastomer composition is curable in the presence of oxygen.

19. The elastomer composition of claim 16, wherein the at least one elastomer is selected from the group consisting of natural rubber, fluoroelastomers, nitrile rubber (NBR), carboxylated nitrile rubber (XNBR), styrene butadiene rubber (SBR), synthetic polyisoprene rubber (IR), neoprene rubber (CR), and a combination thereof.

20. The elastomer composition of claim 16, wherein the at least one peroxide is selected from the group consisting of t-butyl peroxy-2-ethylhexanoate, OO-t-amyl-O-(2-ethylhexyl) monoperoxycarbonate, 1,1-di-(t-amylperoxy)cyclohexane, dibenzoyl peroxide and a combination thereof.

21. The elastomer composition of claim 16, wherein the at least one compound having secondary amine functionality comprises arginine or folic acid.

22. An elastomeric article comprising a cured elastomer composition of claim 16.

23. A process for curing a latex elastomeric mixture, said process comprising:
curing a latex elastomeric mixture in the presence of oxygen,
wherein the latex elastomeric mixture comprises at least one latex elastomer, at least one peroxide, and at least one compound having secondary amine functionality selected from amino acids folic acids, and organic secondary amines.

24. The process of claim 23, wherein the at least one compound having secondary amine functionality is selected from amino acids, folic acid and polyethyleneamines.

25. The process of claim 23 further comprising mixing the at least one elastomer, the at least one peroxide, and the at least one compound having secondary amine functionality to provide the elastomeric mixture, wherein the amounts of the at least one peroxide and the at least one compound having secondary amine functionality are selected such that the elastomeric mixture is curable in the presence of oxygen.

26. The process of claim 23 comprising curing the latex elastomeric mixture in the presence of oxygen at one or more temperatures between 70° C. and 150° C.

27. The process of claim 23 wherein the process occurs at least in part on a mold to form a dip-molded article.

28. The process of claim 23, wherein the at least one compound having secondary amine functionality comprises arginine or folic acid.

29. A dip-molded latex elastomer composition prepared by the process of claim 27.

30. A glove prepared by the process of claim 23.

31. A balloon prepared by the process of claim 23.

32. A condom prepared by the process of claim 23.
